(19) United States
(12) Patent Application Publication
(10) Pub. No.: US 2022/0313609 A1
(43) Pub. Date: Oct. 6, 2022

(54) Nano composite material aiming at acidic sealing zone in osteoclasts and preparation method thereof

(71) Applicant: SIR RUN RUN SHAW HOSPITAL ZHEJIANG UNIVERSITY SCHOOL OF MEDICINE, Hangzhou, Zhejiang (CN)

(72) Inventors: Xianfeng Lin, Hangzhou, Zhejiang (CN); Chenhui Gu, Hangzhou, Zhejiang (CN); Qingqing Wang, Hangzhou, Zhejiang (CN); Shunwu Fan, Hangzhou, Zhejiang (CN)

(21) Appl. No.: 17/426,535

(22) PCT Filed: Apr. 10, 2020
(86) PCT No.: PCT/CN2020/084300

§ 371 Date: Jul. 28, 2021

(51) Int. Cl. A61K 9/127; A61K 47/24; A61K 47/10; A61K 31/65; A61K 33/00; A61P 19/10

(52) U.S. Cl. CPC A61K 9/1273; A61K 47/24; A61K 47/10; A61K 31/65; A61K 33/00; A61P 19/10

(57) ABSTRACT

A nano composite material aiming at an acidic sealing zone in osteoclasts and a preparation method thereof are provided. The nano composite material aiming at the acidic sealing zone in the osteoclasts includes a nano material, bone-targeting molecules, and a compound able to react with the acidic sealing zone in the osteoclasts, wherein: after being modified by the bone-targeting molecules, the nano material is loaded with the compound able to react with the acidic sealing zone in the osteoclasts. Through accurate mature osteoclast targeting and chemically regulated biocascade effects, the osteoclasts are inhibited, which provides a new idea and a new tool for drug therapy of abnormal osteoclast activation.

### BACKGROUND OF THE PRESENT INVENTION

### Field of Invention

The present invention relates to a field of drug therapy for bone tissues, and more particularly to a nano composite material aiming at an acidic sealing zone in osteoclasts and a preparation method thereof.

### Description of Related Arts

Osteoporosis is a global chronic disease characterized by severe bone loss and bone fracture, which brings pains to the patients and greatly reduces the life quality. The bone tumor and the abnormal osteoclast activation during the bone metastasis of the tumor can also cause the abnormal osteoclast activation, thereby causing osteoporosis and pathological bone fracture. The conventional treatment means for the abnormal osteoclast activation comprise the bone resorption inhibitors such as the calcium preparation, vitamin D, calcitonin, bisphosphonate and estrogen, and the bone formation promoters such as the fluoride, anabolic steroid, and parathyroid hormone. Although the above treatment means can affect the function of the osteoclasts or facilitate bone formation to slow down the disease progression, the bone loss cannot be completely inhibited as the acidification and bone destructions caused by the osteoclasts are irreversible. The acidification of the contact interface of the osteoclasts with the bones is the root cause of dissolution and organic degradation of the bone minerals during osteoporosis. Thus, it is urgent to develop a material aiming at the acidic sealing zone in the osteoclasts, so as to prevent and treat the abnormal osteoclast activation.

There are numerous nano materials can be used in drug therapy of the bone tissues, such as the liposomes, polymer nano particles, silicon dioxide particles and nano coatings. Through the specific targeting way and drug release way, these nano materials can achieve the therapeutic effect. When treating osteoporosis, these nano materials can reach the targeted bone tissues and affect the function of the osteoclasts in a certain way. However, the common materials have the problems of inaccurate targeting, insignificant effect, and great toxic and side effects. The Chinese patent application of CN 201710283530.5 disclosed a preparation method of a dual-targeting drug-loaded nano-particle lipid-polymer for osteoporosis, which enhances the targeting effect of the drug to decrease the side effects of the drug. The Chinese patent application of CN 201710841290.6 disclosed an application of a pH-responsive nano material in preparation of a bone resorption inhibitor for preventing and treating osteoporosis, which selectively inhibits the osteoclasts with the pH-responsive graphene oxide, chitosan or hydrogel. The above patent applications optimize the delivery or release process to a certain extent.

However, it is difficult for the conventional materials to solve the two problems at the same time, namely accuracy of osteoclast targeting and physiology safety of the drug. During the progression of osteoporosis, the bone destructions caused by bone resorption of the mature osteoclasts as well as the bone repair and homeostasis maintaining mediated by other cells are important. Thus, the rational materials for treating osteoporosis should be able to target the bone tissues, especially the acidified osteoclasts, in the circulation. Meanwhile, the materials used for targeting and pH response should be materials widely applied in clinic or common materials in the body. If the above two requirements are met, inhibition on the osteoclasts can be realized, while toxic and side effects on other cells can be as small as possible, so as to achieve better resistance to bone resorption and better facilitation on bone formation.

### SUMMARY OF THE PRESENT INVENTION

Aiming at deficiencies in prior art, the present invention provides a nano composite material aiming at an acidic sealing zone in osteoclasts and a preparation method thereof. Through bone targeting with a common clinical drug, accurate targeting and function inhibition of the osteoclasts are both realized by a chemical reaction.

In order to accomplish the above object, the present invention adopts technical solutions as follows.

A nano composite material aiming at an acidic sealing zone in osteoclasts comprises a nano material, bone-targeting molecules, and a compound able to react with the acidic sealing zone in the osteoclasts, wherein: after being modified by the bone-targeting molecules, the nano material is loaded with the compound able to react with the acidic sealing zone in the osteoclasts; the nano material is loadable and modifiable; the bone-targeting molecules have an obvious affinity to bone tissues; and the compound able to react with the acidic sealing zone in the osteoclasts is alkalescent or neutral bicarbonate.

Preferably, the nano material is liposomes, polymer nano particles or mesoporous silicon oxide particles.

Preferably, the bone-targeting molecules are tetracycline, phosphonate or aspartic acid polypeptide sequences.

Preferably, the compound able to react with the acidic sealing zone in the osteoclasts is sodium bicarbonate, potassium bicarbonate or ammonium bicarbonate; further preferably, the compound is sodium bicarbonate having a concentration of 1 mol/L.

A method for preparing the nano composite material aiming at the acidic sealing zone in the osteoclasts comprises steps of: cross-linking the loadable and modifiable nano material with the bone-targeting molecules; dissolving in chloroform with lecithin and cholesterol, and controlling a pH value to 8.0-8.4; at a room temperature, magnetically stirring and cross-linking for 24-72 hours; forming a membrane in a rotary evaporator; adding a solution to be loaded, and shaking for hydration; ultrasonically emulsifying, and dialyzing; wherein: a molar ratio of the loadable and modifiable nano material to the bone-targeting molecules is 1:1-1:2.

Preferably, a functionalized molecule in the loadable and modifiable nano material is functionalized phospholipid; after cross-linking with the bone-targeting molecules, bone-targeting functionalized phospholipid is obtained.

Preferably, the step of “ultrasonically emulsifying” specifically comprises steps of: turning on for 1-2 seconds with a power of 30-70%, then turning off for 2-3 seconds, and repeating for 5-20 minutes; and the step of “dialyzing” lasts for 1-3 days.

Preferably, the functionalized phospholipid is DSPE-PEG-NHS; and the bone-targeting molecules are tetracycline, namely TC.

The present invention has beneficial effects as follows. The present invention provides the nano composite material aiming at the acidic sealing zone in the osteoclasts and the preparation method thereof. Through accurate mature osteoclast targeting and chemically regulated biocascade effects, the osteoclasts are inhibited, which provides a new idea and a new tool for drug therapy of abnormal osteoclast activation.

Compared with the conventional drug or material for treating osteoporosis, the present invention achieves significant progresses as follows.

1) Accurate dual-targeting is conducted with the bone tissue-targeting molecules and the pH response aiming at the acidic sealing zone in the osteoclasts, so that the utilization of the drug is improved and the side effects on other tissues and organs are reduced.

2) The used drug is the physiological compound existing in the human body, with the low toxicity. It can serve as the component generating the therapeutic effect and the component of rapid pH response at the same time, so that dual functions are achieved.

3) The resistance to osteoclastic bone erosion is verified by the in-vitro experiment. The present invention has the obvious inhibitory effects on the count and area of the osteoclasts, the resorbed count and the resorbed area.

4) The facilitation on formation of the exosomes by the osteoclasts is verified by the in-vitro experiment. With utilizing the receptor activator of nuclear factor-kappa B (RANK) on the surface of the exosomes, ineffective binding with the receptor activator of nuclear factor-kappa B ligand (RANKL) in serum is realized, thereby realizing the long-term inhibitory effects on the osteoclasts.

5) The resistance to osteoporosis is verified by the in-vivo experiment. The present invention has the significant improvements on bone volume per tissue volume, trabecular number, and trabecular separation.

6) As a model of the nano composite material aiming at the acidic sealing zone in the osteoclasts, each component is replaceable, which has the great referential significance.

In conclusion, the nano composite material aiming at the acidic sealing zone in the osteoclasts, provided by the present invention, can be applied in preventing and treating the abnormal activation of the osteoclasts, showing the obvious therapeutic effect.

### BRIEF DESCRIPTION OF THE DRAWINGS

The patent of application file contains at least one drawing executed in color. Copies of this patent or patent application publication with color drawing(s) will be provided by the Office upon request and payment of the necessary fee.

In order to more clearly illustrate objects, technical solutions and beneficial effects of the present invention, the accompanying drawings are described as follows.

FIG. 1(a)-FIG. 1(g) show preparation and representation of a nano composite material aiming at an acidic sealing zone in osteoclasts according to the present invention, wherein: FIG. 1(a) is a sketch view of an action mechanism of the nano composite material aiming at the acidic sealing zone in the osteoclasts; FIG. 1(b) and FIG. 1(c) are sketch views of a preparation process of the nano composite material aiming at the acidic sealing zone in the osteoclasts; FIG. 1(d)-FIG. 1(f) show verification of cross-linking between bone-targeting molecules and functionalized phospholipid respectively through a matrix-assisted laser desorption/ionization time of flight (MALDI-TOF) mass spectrometer, a laser scanning confocal microscope and a fluorescent spectrophotometer; and FIG. 1(g) shows representation of the nano composite material aiming at the acidic sealing zone in the osteoclasts under a cryo-transmission electron microscope.

FIG. 2(a)-FIG. 2(i) show verification of functions of the nano composite material aiming at the acidic sealing zone in the osteoclasts according to the present invention, wherein: FIG. 2(a) shows verification of an acid resistance of the nano composite material aiming at the acidic sealing zone in the osteoclasts by an acidometric titration experiment; FIG. 2(b) shows verification of a pH response of the nano composite material aiming at the acidic sealing zone in the osteoclasts by measuring a particle size under different pH values; FIG. 2(c) and FIG. 2(d) show verification of mechanical changes of the nano composite material aiming at the acidic sealing zone in the osteoclasts under different pH values through an in-situ liquid atomic force microscope; FIG. 2(e) show verification of a release of the nano composite material aiming at the acidic sealing zone in the osteoclasts under an acidic condition (pH=4) through the cryo-transmission electron microscope; FIG. 2(f) and FIG. 2(g) show verification of long-term (7 days) stability and rapid pH response of the nano composite material aiming at the acidic sealing zone in the osteoclasts through an in-vitro fluorescence microscope; FIG. 2(h) show verification of a rapid enrichment effect of the nano composite material aiming at the acidic sealing zone in the osteoclasts in targeted bones of mice through in-vivo fluorescence; and FIG. 2(i) show verification of an inhibitory effect of the nano composite material aiming at the acidic sealing zone in the osteoclasts on osteoclastic bone erosion through a fluorescence confocal microscope.

FIG. 3(a)-FIG. 3(k) show inhibitory effects of the nano composite material aiming at the acidic sealing zone on the osteoclasts through chemically regulated biocascade effects according to the present invention, wherein: FIG. 3(a) show verification of an inhibitory effect of the nano composite material aiming at the acidic sealing zone on the osteoclasts through tartrate resistant acid phosphatase (TRAP)-staining; FIG. 3(b) show verification of a significant improvement of the nano composite material aiming at the acidic sealing zone in the osteoclasts on the osteoclastic bone erosion through the scanning electron microscope; FIG. 3(c) and FIG. 3(d) show verification of inhibition of the nano composite material aiming at the acidic sealing zone in the osteoclasts on an increment effect of NFATc-1, c-Fos and CTSK expressions in the osteoclasts with time through Western-blot and quantitative polymerase chain reaction (q-PCR); FIG. 3(e) show verification of an inhibitory effect of the nano composite material aiming at the acidic sealing zone in the osteoclasts on formation of the acidic sealing zone in the osteoclasts through the laser scanning confocal microscope; FIG. 3(f) and FIG. 3(g) show verification of inhibition of the nano composite material aiming at the acidic sealing zone in the osteoclasts on an increment effect of receptor activator of nuclear factor-kappa B (RANK) expressions in the osteoclasts with time through Western-blot and the fluorescence confocal microscope; FIG. 3(h) and FIG. 3(i) show verification of facilitation of the nano composite material aiming at the acidic sealing zone in the osteoclasts on formation of RANK-containing exosomes by the osteoclasts through Western-blot and extracellular vesicle flow cytometry; FIG. 3(j) and FIG. 3(k) show verification of an inhibitory effect of RANK-containing extracellular vesicles secreted by the osteoclasts on formation of the osteoclasts through TRAP staining, wherein secretion of the RANK-containing extracellular vesicles is facilitated by the nano composite material aiming at the acidic sealing zone in the osteoclasts.

FIG. 4(a)-FIG. 4(f) show inhibitory effects of the nano composite material aiming at the acidic sealing zone in the osteoclasts on osteoporosis of ovariectomy (OVX) mice according to the present invention, wherein: FIG. 4(a) shows establishment of an animal model, and grouping and evaluation ways; FIG. 4(b) and FIG. 4(c) show verification of significant improvements of the nano composite material aiming at the acidic sealing zone in the osteoclasts on bone volume per tissue volume, trabecular number, and trabecular separation of vertebral body, femur and tibia of the OVX mice through micro-CT; FIG. 4(d) and FIG. 4(e) show verification of significant improvements of the nano composite material aiming at the acidic sealing zone in the osteoclasts on bone volume per tissue volume, number of the osteoclasts, and surface area of the osteoclasts of bone tissues of the OVX mice through hematoxylin-eosin (H&E) staining and TRAP staining; and FIG. 4(f) show verification of a significant inhibitory effect of the nano composite material aiming at the acidic sealing zone in the osteoclasts on osteoclast metabolic indicators of the OVX mice through serological indicators.

### DETAILED DESCRIPTION OF THE PREFERRED EMBODIMENT

The present invention provides a nano composite material aiming at an acidic sealing zone in osteoclasts and a preparation method thereof. The present invention will be described in detail with the following examples, but these examples cannot be understood as the limitation to the protection scope of the present invention.

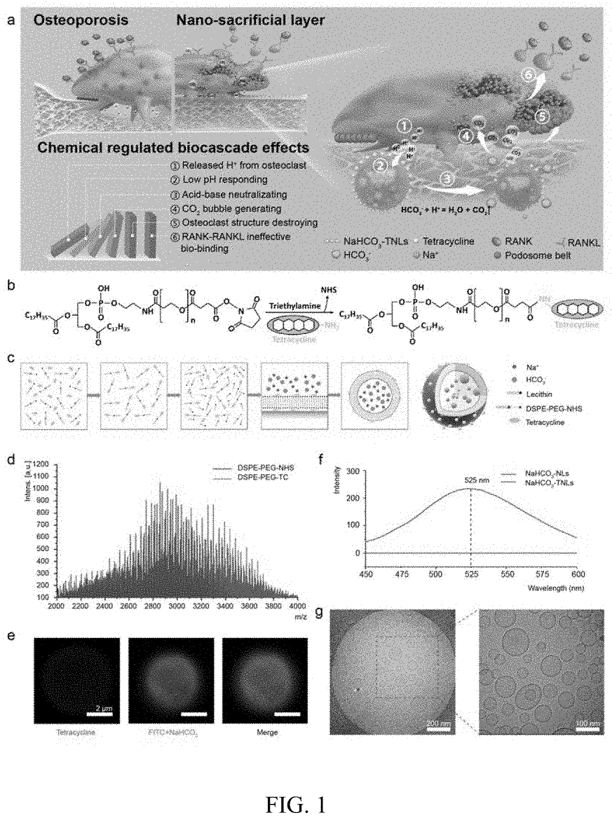

A sketch view of an action mechanism of the nano composite material aiming at the acidic sealing zone in the osteoclasts is shown in FIG. 1(a).

According to the present invention, the nano composite material aiming at the acidic sealing zone in the osteoclasts is sodium bicarbonate-loaded and tetracycline-modified nano liposomes (NaHCO₃-TNLs for short), and prepared through steps of: dissolving 20.00 mg DSPE-PEG-NHS and 3.05 mg tetracycline in 10.00 mL chloroform; adding triethylamine, and regulating a pH value to 8.2; at a room temperature, magnetically stirring and cross-linking for 48 hours, and then obtaining DSPE-PEG-TC; dissolving DSPE-PEG-TC in chloroform with 80.00-120.00 mg lecithin and 12.00-20.00 mg cholesterol; forming a membrane in a rotary evaporator; adding 10 mL sodium bicarbonate solution having a concentration of 1 mol/L, and shaking for hydration; ultrasonically emulsifying, specifically comprising steps of turning on for 2 seconds with a power of 40%, then turning off for 3 seconds, and repeating for 10 minutes; dialyzing in a dialysis bag for 72 hours; taking out, and filtering with a filter head of 0.22 μm; and preserving at 4° C. The prepared material is shown in FIG. 1(b) and FIG. 1(c).

The present invention is able to prepare other nano composite materials aiming at the acidic sealing zone in the osteoclasts, such as the nano liposomes loaded with ammonium bicarbonate or potassium bicarbonate and modified by tetracycline or alendronic acid, which can achieve same technical effects.

### EXAMPLE 1

### Preparation of sodium bicarbonate-loaded and tetracycline-modified Nano Liposomes

The preparation process comprises steps of:

(1) dissolving 20.00 mg DSPE-PEG-NHS and 3.05 mg tetracycline in 10.00 mL chloroform; adding triethylamine, and regulating a pH value to 8.2; at a room temperature, magnetically stirring and cross-linking for 48 hours;

(2) dissolving a product obtained in the step (1) in chloroform with 100.00 mg lecithin and 16.00 mg cholesterol; and forming a membrane in a rotary evaporator;

(3) adding 10 mL sodium bicarbonate solution having a concentration of 1 mol/L into a flask, and shaking for hydration;

(4) ultrasonically emulsifying, specifically comprising steps of: turning on for 2 seconds with a power of 40%, then turning off for 3 seconds, and repeating for 10 minutes; and

(5) dialyzing in a dialysis bag for 72 hours; taking out, and filtering with a filter head of 0.22 μm; and preserving at 4° C.

### EXAMPLE 2

### Preparation of sodium bicarbonate-loaded and alendronic acid-modified Nano Liposomes

The preparation process comprises steps of:

(1) dissolving 20.00 mg DSPE-PEG-NHS and 2.30 mg alendronate sodium in 10.00 mL chloroform; adding triethylamine, and regulating a pH value to 8.2; at a room temperature, magnetically stirring and cross-linking for 48 hours;

(2) dissolving a product obtained in the step (1) in chloroform with 100.00 mg lecithin and 16.00 mg cholesterol; and forming a membrane in a rotary evaporator;

(3) adding 10 mL sodium bicarbonate solution having a concentration of 1 mol/L into a flask, and shaking for hydration;

(4) ultrasonically emulsifying, specifically comprising steps of: turning on for 2 seconds with a power of 40%, then turning off for 3 seconds, and repeating for 20 minutes; and

(5) dialyzing in a dialysis bag for 72 hours; taking out, and filtering with a filter head of 0.22 μm; and preserving at 4° C.

### EXAMPLE 3

### Preparation of potassium bicarbonate-loaded and tetracycline-modified Nano Liposomes

The preparation process comprises steps of:

(1) dissolving 20.00 mg DSPE-PEG-NHS and 3.05 mg tetracycline in 10.00 mL chloroform; adding triethylamine, and regulating a pH value to 8.2; at a room temperature, magnetically stirring and cross-linking for 24 hours;

(2) dissolving a product obtained in the step (1) in chloroform with 80.00 mg lecithin and 16.00 mg cholesterol; and forming a membrane in a rotary evaporator;

(3) adding 10 mL potassium bicarbonate solution having a concentration of 1 mol/L into a flask, and shaking for hydration;

(4) ultrasonically emulsifying, specifically comprising steps of: turning on for 2 seconds with a power of 40%, then turning off for 3 seconds, and repeating for 10 minutes; and

(5) dialyzing in a dialysis bag for 72 hours; taking out, and filtering with a filter head of 0.22 μm; and preserving at 4° C.

### EXAMPLE 4

### Preparation of potassium bicarbonate-loaded and alendronic acid-modified Nano Liposomes

The preparation process comprises steps of:

(1) dissolving 20.00 mg DSPE-PEG-NHS and 2.30 mg alendronate sodium in 10.00 mL chloroform; adding triethylamine, and regulating a pH value to 8.2; at a room temperature, magnetically stirring and cross-linking for 48 hours;

(2) dissolving a product obtained in the step (1) in chloroform with 120.00 mg lecithin and 16.00 mg cholesterol; and forming a membrane in a rotary evaporator;

(3) adding 10 mL potassium bicarbonate solution having a concentration of 1 mol/L into a flask, and shaking for hydration;

(4) ultrasonically emulsifying, specifically comprising steps of: turning on for 2 seconds with a power of 40%, then turning off for 3 seconds, and repeating for 5 minutes; and

(5) dialyzing in a dialysis bag for 72 hours; taking out, and filtering with a filter head of 0.22 μm; and preserving at 4° C.

### EXAMPLE 5

### Preparation of ammonium bicarbonate-loaded and tetracycline-modified Nano Liposomes

The preparation process comprises steps of:

(1) dissolving 20.00 mg DSPE-PEG-NHS and 3.05 mg tetracycline in 10.00 mL chloroform; adding triethylamine, and regulating a pH value to 8.2; at a room temperature, magnetically stirring and cross-linking for 48 hours;

(2) dissolving a product obtained in the step (1) in chloroform with 100.00 mg lecithin and 12.00 mg cholesterol; and forming a membrane in a rotary evaporator;

(3) adding 10 mL ammonium bicarbonate solution having a concentration of 1 mol/L into a flask, and shaking for hydration;

(4) ultrasonically emulsifying, specifically comprising steps of: turning on for 1 second with a power of 40%, then turning off for 3 seconds, and repeating for 10 minutes; and

(5) dialyzing in a dialysis bag for 24 hours; taking out, and filtering with a filter head of 0.22 μm; and preserving at 4° C.

### EXAMPLE 6

### Preparation of ammonium bicarbonate-loaded and alendronic acid-modified Nano Liposomes

The preparation process comprises steps of:

(1) dissolving 20.00 mg DSPE-PEG-NHS and 2.30 mg alendronate sodium in 10.00 mL chloroform; adding triethylamine, and regulating a pH value to 8.2; at a room temperature, magnetically stirring and cross-linking for 48 hours;

(2) dissolving a product obtained in the step (1) in chloroform with 100.00 mg lecithin and 20.00 mg cholesterol; and forming a membrane in a rotary evaporator;

(3) adding 10 mL ammonium bicarbonate solution having a concentration of 1 mol/L into a flask, and shaking for hydration;

(4) ultrasonically emulsifying, specifically comprising steps of: turning on for 2 seconds with a power of 70%, then turning off for 2 seconds, and repeating for 10 minutes; and

(5) dialyzing in a dialysis bag for 72 hours; taking out, and filtering with a filter head of 0.22 μm; and preserving at 4° C.

Composite Evaluation of NaHCO₃-TNLs in Example 1

1. Through respectively detecting mass spectrums of DSPE-PEG-NHS and DSPE-PEG-TC with the matrix-assisted laser desorption/ionization time of flight (MALDI-TOF) mass spectrometer, it is obtained that: a molecular weight of DSPE-PEG-NHS is distributed at 2900 and a molecular weight of DSPE-PEG-TC is distributed at 3250, which are consistent with theoretical molecular weights thereof (as shown in FIG. 1(d)).

2. Fluorescein isothiocyanate (FITC) and sodium bicarbonate solution are co-loaded; through a laser scanning confocal microscope, it can be seen that localization of the tetracycline fluorescence and the liposome membrane are consistent (as shown in FIG. 1(e)).

3. The fluorescent spectrophotometer shows that the fluorescence intensity of NaHCO₃-TNLs is obviously increased at 525 nm in comparison to NaHCO₃-NLs without tetracycline modification (as shown in FIG. 1(f)).

4. The cryo-transmission electron microscope shows that the particle size of NaHCO₃-TNLs is nano-scaled and morphology of NaHCO₃-TNLs is uniform.

Characteristic Evaluation of NaHCO₃-TNLs in Example 1

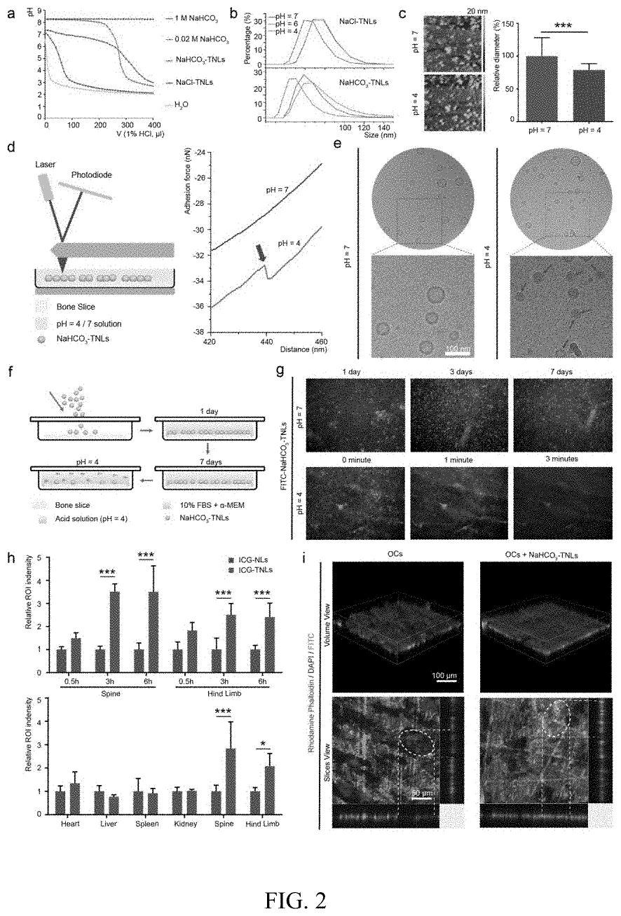

1. A titration experiment is conducted on the sodium bicarbonate-loaded and tetracycline-modified nano liposomes (NaHCO₃-TNLs), the sodium chloride-loaded and tetracycline-modified nano liposomes (NaCl-TNLs), water, the sodium bicarbonate solution having a concentration of 1 mol/L, and the sodium bicarbonate solution having a concentration of 0.02 mol/L with 1% hydrochloric acid, and a dynamic change of the pH value thereof is detected in real time. The results show that NaHCO₃-TNLs have the excellent acid resistance (as shown in FIG. 2(a)).

2. The particle sizes of NaHCO₃-TNLs and NaCl-TNLs are measured respectively under the pH values of 7, 6 and 4. The results show that the particle size of NaHCO₃-TNLs is obviously decreased in an acid environment of PH=4, indicating that the contents are released in the acid environment (as shown in FIG. 2(b)).

3. The mechanical characteristics of NaHCO₃-TNLs under the pH values of 7 and 4 are compared through an in-situ liquid atomic force microscope. The results show that: the particle size of NaHCO₃-TNLs is obviously decreased under the pH value of 4, and the liposome membrane tends to rupture (as shown in FIG. 2(c) and FIG. 2(d)).

4. The morphologies of NaHCO₃-TNLs under the pH values of 7 and 4 are compared through the cryo-transmission electron microscope. The results show that rupture of the liposome membrane occurs under the pH value of 4 (as shown in FIG. 2(e)).

5. FITC is loaded into NaHCO₃-TNLs and thereafter incubated in a 10% serum medium with bovine bone slices, and the fluorescence respectively 1 day, 3 days and 7 days after incubation is observed; 7 days later, the medium is placed in an environment with the pH value of 4, and the liposome fluorescence respectively 0 minute, 1 minute and 3 minutes after placement is observed. The results show that: NaHCO₃-TNLs can be adsorbed on the bone surface in 7 days and keep stable; moreover, NaHCO₃-TNLs still have the rapid pH response function 7 days later.

6. Indocyanine green-loaded and tetracycline-modified nano liposomes (ICG-TNLs) and indocyanine green-loaded nano liposomes without tetracycline modification (ICG-NLs) are injected into the caudal veins of mice with a dosage of 0.025 ml/g. The results show that: compared with ICG-NLs, ICG-TNLs have a significant rapid enrichment effect on bone tissues (as shown in FIG. 2(h)).

7. NaHCO₃-TNLs and NaCl-TNLs are respectively co-incubated with the FITC-coated bovine bone slices, and the mature osteoclasts are respectively implanted thereon. The results show that the FITC fluorescence intensity and area of the bone surface with NaHCO₃-TNLs are significantly better than that with NaCl-TNLs, indicating that NaHCO₃-TNLs can effectively inhibit the bone erosion effect of the osteoclasts (as shown in FIG. 2(i)).

Inhibitory Effects of NaHCO₃-TNLs in Example 1 on Osteoclasts

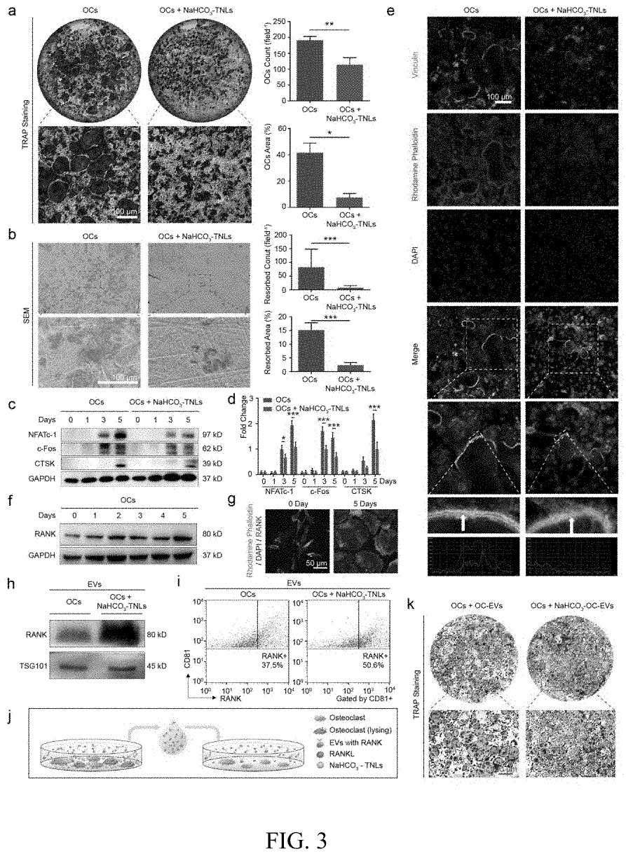

1. NaHCO₃-TNLs are added into an osteoclast inducing system and compared with a pure osteoclast inducing system. The results obtained through tartrate resistant acid phosphatase (TRAP)-staining show that the count and the area of the osteoclasts are both greatly inhibited with NaHCO₃-TNLs, indicating that NaHCO₃-TNLs have the significant inhibitory effect on the osteoclasts (as shown in FIG. 3(a)).

2. NaHCO₃-TNLs are added into an osteoclast inducing system cultured with the bovine bone slices and compared with a pure osteoclast inducing system cultured with the bovine bone slices. The results obtained through the scanning electron microscope show that an absorption count and an absorption area are both greatly inhibited with NaHCO₃-TNLs, indicating that NaHCO₃-TNLs have the significant inhibitory effect on the osteoclasts (as shown in FIG. 3(b)).

3. NaHCO₃-TNLs are added into the osteoclast inducing system and compared with the pure osteoclast inducing system. The results obtained through Western-blot and quantitative polymerase chain reaction (q-PCR) show that NaHCO₃-TNLs can inhibit an increment effect of NFATc-1, c-Fos and CTSK expressions in the osteoclasts with time; the results obtained through the laser scanning confocal microscope show that NaHCO₃-TNLs can inhibit formation of the actin ring of the osteoclasts, indicating that NaHCO₃-TNLs have the significant inhibitory effect on the bone resorption function of the osteoclasts (as shown in FIG. 3(c)-FIG. 3(e)).

4. The receptor activator of nuclear factor-kappa B (RANK) expressions in the osteoclast inducing system are evaluated every day. The results obtained through Western-blot and the laser scanning confocal microscope show that the RANK expression quantity in the osteoclasts is progressively increased with maturation of the osteoclasts (as shown in FIG. 3(f) and FIG. 3(g)).

5. NaHCO₃-TNLs are added into the osteoclast inducing system and compared with the pure osteoclast inducing system, and the exosomes are extracted for evaluation. The results obtained through Western-blot and exosome flow cytometry show that the RANK content in the extracellular vesicles is greatly increased with NaHCO₃-TNLs. Compared with the above results, it can be known that NaHCO₃-TNLs can facilitate the osteoclasts to secrete the RANK-containing extracellular vesicles (as shown in FIG. 3(h) and FIG. 3(i)).

6. The extracellular vesicles extracted above are respectively added into the osteoclast inducing systems. The results obtained through TRAP-staining show that the osteoclasts are greatly inhibited with NaHCO₃-TNLs induced extracellular vesicles, indicating that the RANK-containing extracellular vesicles can further inhibit the osteoclasts (as shown in FIG. 3(j) and FIG. 3(k)).

Therapeutic Effects of NaHCO₃-TNLs in Example 1 on Osteoporosis of Ovariectomy (OVX) Mice

1. Establishing Animal Disease Model and Grouping

Grouping: The 11-week-old C57BL/6 female mice are divided into four groups, wherein: for mice of the first group (Sham), a sham operation is conducted, and normal saline is injected into the caudal veins; for mice of the second group (OVX), the ovary is removed, and normal saline is injected into the caudal veins; for mice of the third group (OVX+NaCl-TNLs), the ovary is removed, and NaCl-TNLs are injected into the caudal veins; for mice of the fourth group (OVX+NaHCO₃-TNLs), the ovary is removed, and NaHCO₃-TNLs are injected into the caudal veins.

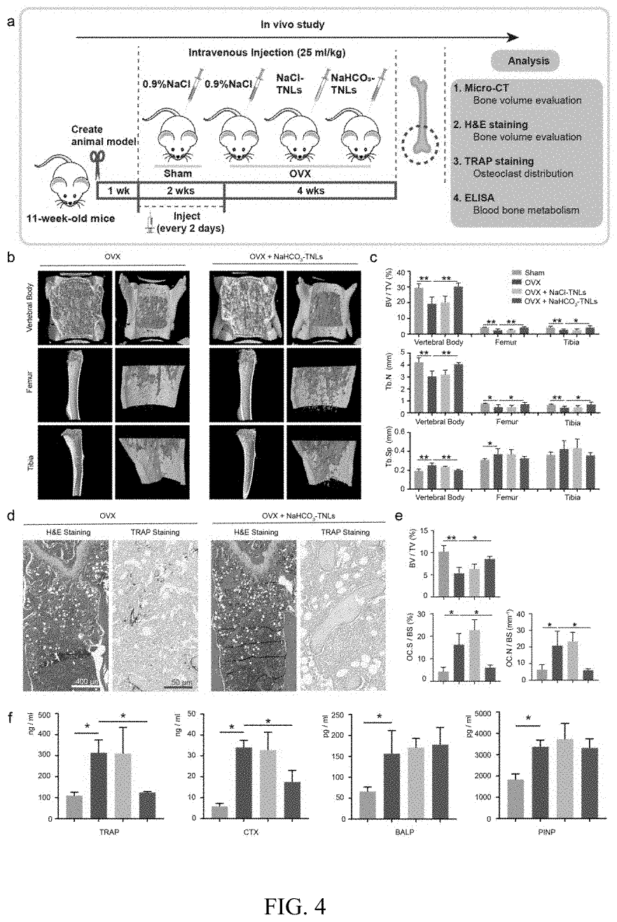

Implementation: Corresponding operations are conducted on each group of mice; one week later, corresponding drugs with a dosage of 0.025 ml/g are injected into the caudal veins every two days, lasting for 2 weeks; 4 weeks after administration is finished, the vertebral body, femur, tibia and blood of each group of mice are taken out for analysis. The analysis methods comprise micro-CT, hematoxylin-eosin (H&E) staining, TRAP staining, and enzyme-linked immunosorbent assay (ELISA) of serum bone metabolic indicators (as shown in FIG. 4(a)).

2. The micro-CT results of the vertebral body, femur and tibia of each experimental group are compared. The results show that the factors of bone volume per tissue volume (BV/TV), trabecular number (Tb. N) and trabecular separation (Tb. Sp) of the OVX+NaHCO₃-TNLs group are all significantly better than that of the OVX group (as shown in FIG. 4(b) and FIG. 4(c)).

3. The H&E staining and TRAP staining results of the vertebral body, femur and tibia of each experimental group are compared. The results show that factors of BV/TV, surface area of the osteoclasts on the bone surface (OC. S/BS), and number of the osteoclasts on the bone surface (OC. N/BS) of the OVX+NaHCO₃-TNLs group are all significantly better than that of the OVX group (as shown in FIG. 4(d) and FIG. 4(e)).

4. The serum bone metabolic indicators of each experimental group are compared. The results show that the osteoclasts metabolic indicators of the OVX+NaHCO₃-TNLs group are significantly lower than that of the OVX group. Combined with the above experimental results, it is indicated that NaHCO₃-TNLs can effectively treat bone loss and osteoclast metabolism of the OVX mice, so as to treat osteoporosis (as shown in FIG. 4(f)).

For the nano composite materials aiming at the acidic sealing zone in the osteoclasts obtained through the examples 2-6, composite evaluation, characteristic evaluation, evaluation of the inhibitory effects on the osteoclasts, and evaluation of the therapeutic effects on osteoporosis of the OVX mice are respectively conducted. The obtained results are similar to the results of NaHCO₃-TNLs in the example 1, indicating that: through the adjustment of reagent concentration and treatment time determined by above optimizations, the preparation of the nano composite material aiming at the acidic sealing zone in the osteoclasts, having the similar effect, can be realized.

It can be known from the above examples that: through targeting the bone tissues, the nano composite material aiming at the acidic sealing zone in the osteoclasts provided by the present invention can conduct an aerogenic pH response with the acidic sealing zone in the osteoclasts, and destroy the acidic sealing zone in the osteoclasts while neutralizing acidification, so that maturation of the osteoclasts is inhibited, the osteoclasts are facilitated to secrete the RANK-containing extracellular vesicles, and ineffective binding is formed with receptor activator of nuclear factor-kappa B ligand (RANKL) in serum, thereby achieving the long-term therapeutic effects on the abnormal osteoclast activation.

The above-described is only the examples of the present invention. Although the present invention is described in detailed with the above examples, one of ordinary skill in the art should understand that various improvements and modifications can be made without departing from the principle of the present invention. These improvements and modifications should be all encompassed in the protection scope of the present invention and in the scope limited by the claims of the present invention.

1. A nano composite material aiming at an acidic sealing zone in osteoclasts, comprising a nano material, bone-targeting molecules, and a compound able to react with the acidic sealing zone in the osteoclasts, wherein: after being modified by the bone-targeting molecules, the nano material is loaded with the compound able to react with the acidic sealing zone in the osteoclasts; the nano material is loadable and modifiable; the bone-targeting molecules have an obvious affinity to bone tissues; and the compound able to react with the acidic sealing zone in the osteoclasts is alkalescent or neutral bicarbonate.

2. The nano composition material, as recited in claim 1, wherein: the nano material is liposomes, polymer nano particles or mesoporous silicon oxide particles.

3. The nano composite material, as recited in claim 1, wherein: the bone-targeting molecules are tetracycline, phosphonate or aspartic acid polypeptide sequences.

4. The nano composite material, as recited in claim 1, wherein: the compound able to react with the acidic sealing zone in the osteoclasts is sodium bicarbonate, potassium bicarbonate or ammonium bicarbonate.

5. The nano composite material, as recited in claim 1, wherein: the compound able to react with the acidic sealing zone in the osteoclasts is sodium bicarbonate having a concentration of 1 mol/L.

6. A method for preparing the nano composite material aiming at the acidic sealing zone in the osteoclasts as recited in claim 1, comprising steps of: cross-linking the loadable and modifiable nano material with the bone-targeting molecules; dissolving in chloroform with lecithin and cholesterol, and controlling a pH value to 8.0-8.4; at a room temperature, magnetically stirring and cross-linking for 24-72 hours; forming a membrane in a rotary evaporator; adding a solution to be loaded, and shaking for hydration; ultrasonically emulsifying, and dialyzing; wherein: a molar ratio of the loadable and modifiable nano material to the bone-targeting molecules is 1:1-1:2.

7. The method, as recited in claim 6, wherein: a functionalized molecule in the loadable and modifiable nano material is functionalized phospholipid; after cross-linking with the bone-targeting molecules, bone-targeting functionalized phospholipid is obtained.

8. The method, as recited in claim 6, wherein: the step of “ultrasonically emulsifying” specifically comprises steps of: turning on for 1-2 seconds with a power of 30-70%, then turning off for 2-3 seconds, and repeating for 5-20 minutes; and the step of “dialyzing” lasts for 1-3 days.

9. The method, as recited in claim 7, wherein: the functionalized phospholipid is DSPE-PEG-NHS; and the bone-targeting molecules are tetracycline (TC).
